(19) United States
(12) Patent Application Publication
(10) Pub. No.: US 2023/0293099 A1
(43) Pub. Date: Sep. 21, 2023

(54) DRUG INJECTION ADJUSTING APPARATUS AND METHOD USING REINFORCEMENT LEARNING

(71) Applicants: Korea University Research and Business Foundation, Seoul (KR); Seoul National University R&DB Foundation, Seoul (KR)

(72) Inventors: Joongheon KIM, Seoul (KR); Myung Jae SHIN, Seoul (KR); Hyung-Chul LEE, Seoul (KR)

(73) Assignees: Korea University Research and Business Foundation, Seoul (KR); Seoul National University R&DB Foundation, Seoul (KR)

(21) Appl. No.: 18/016,507

(22) PCT Filed: Jun. 2, 2021
(86) PCT No.: PCT/KR2021/006850

§ 371 Date: Jan. 17, 2023

(30) Foreign Application Priority Data
Jul. 14, 2020 (KR) 10-2020-0086957

(51) Int. Cl. A61B 5/00; A61M 5/14

(52) U.S. Cl. CPC A61B 5/4821; A61M 5/14; A61M 2005/14208

(57) ABSTRACT

A drug injection control device generates a policy model by learning a change in anesthetic state information due to a drug injection rate set so that anesthetic state information of a patient follows target anesthetic state information, generates a prediction model by learning a change in the anesthetic state information according to a change in the drug injection rate, sets the drug injection rate from the anesthetic state information based on the policy model, and predicts expected anesthetic state information from the set drug injection rate and a previously set drug injection rate based on the prediction model.

### TECHNICAL FIELD

The present disclosure relates to a device and method for controlling drug injection using reinforcement learning, and more particularly, to a device and method for controlling drug injection for maintaining an anesthetic state of a patient during surgery.

### BACKGROUND ART

In general, an anesthesiologist adjusts a drug injection rate in order to induce a patient's condition into a constant anesthetic state. In modern times, a patient's bispectral index (BIS) or electroencephalogram (EEG) is measured to directly control a drug injection rate to keep the patient's condition constant. In this case, the anesthesiologist uses an infusion pump to inject a drug into the patient.

In this case, in a process of inducting the patient into anesthesia or sedation including hypnosis and analgesia, the anesthesiologist uses a fast-acting drug such as propofol or remifentanil, and in this process, pharmacokinetics (PK)—pharmacodynamics (PD) is used.

Maintaining the patient's condition in a constant anesthetic state is controlled by the anesthesiologist, and thus, it may be difficult to maintain the anesthetic state depending on the anesthesiologist's health condition and skill level. For example, when the patient is under-injected, the patient may regain consciousness during surgery, and when the patient is over-injected, the patient may experience hemodynamic instability and other side effects.

Accordingly, there is a demand for a method of appropriately adjusting a drug injected into a patient so that the patient's anesthetic state is maintained constant.

### DISCLOSURE

### Technical Problem

A technical problem to be solved by the present disclosure is to provide a device and method for controlling drug injection to stably maintain a patient's anesthetic state during surgery using reinforcement learning.

### Technical Solution

According to an aspect of the present disclosure, a drug injection control device includes an anesthetic state information calculation unit configured to calculate anesthetic state information of a patient, a policy model training unit configured to set target anesthetic state information pre-set for the anesthetic state information, calculate a compensation value according to a change in the anesthetic state information due to a drug injection rate set so that the anesthetic state information follows the target anesthetic state information, and generate a policy model by learning the compensation value, a prediction model training unit configured to generate a prediction model, by learning the change in the anesthetic state information according to the drug injection rate, a control unit configured to set the drug injection rate from the anesthetic state information, based on the policy model, and a prediction unit configured to predict expected anesthetic state information from the set drug injection rate and a previously set drug injection rate, based on the prediction model.

Also, the anesthetic state information calculation unit may be configured to calculate an effect-site concentration and a plasma concentration based on the patient's fat-free mass and a drug model pre-provided for a drug injected into the patient, and calculate the anesthetic state information to indicate the patient's condition according to the effect-site concentration and the plasma concentration.

Also, the control unit may be configured to, according to a difference between the anesthetic state information calculated from the patient's condition and the target anesthetic state information, an injection rate of remifentanil injected into the patient during a pre-set time interval, and an injection rate of propofol injected into the patient during the pre-set time interval, control the injection rate of remifentanil and the injection rate of propofol.

Also, the policy model training unit may be configured to calculate a compensation value according to the difference between the target anesthetic state information and the anesthetic state information calculated from the patient's condition changed after the pre-set time interval elapses, after the injection rate of remifentanil and the injection rate of propofol are controlled.

Also, the policy model training unit may be configured to, based on a plurality of compensation values calculated from changes in different pieces of anesthetic state information, from arbitrary anesthetic state information, calculate an expected value according to a plurality of compensation values matched to changes in anesthetic state information in a process where a time interval pre-set for a change in the anesthetic state information elapses several times.

Also, the policy model training unit may be configured to generate the policy model according to a drug injection rate in a change in the anesthetic state information matched to a compensation value selected so that the expected value is calculated as a maximum value.

According to another aspect of the present disclosure, a drug injection control method using a drug injection control device using reinforcement learning includes calculating anesthetic state information of a patient, setting target anesthetic state information pre-set for the anesthetic state information, calculating a compensation value according to a change in the anesthetic state information due to a drug injection rate set so that the anesthetic state information follows the target anesthetic state information, and generating a policy model by learning the compensation value, generating a prediction model, by learning the change in the anesthetic state information according to the drug injection rate, setting the drug injection rate from the anesthetic state information, based on the policy model, and predicting expected anesthetic state information from the set drug injection rate and a previously set drug injection rate, based on the prediction model.

Also, the calculating of the anesthetic state information may include calculating an effect-site concentration and a plasma concentration based on the patient's fat-free mass and a drug model pre-provided for a drug injected into the patient, and calculating the anesthetic state information to indicate the patient's condition according to the effect-site concentration and the plasma concentration.

Also, the setting of the drug injection rate may include, according to a difference between the anesthetic state information calculated from the patient's condition and the target anesthetic state information, an injection rate of remifentanil injected into the patient during a pre-set time interval, and an injection rate of propofol injected into the patient during the pre-set time interval, controlling the injection rate of remifentanil and the injection rate of propofol.

Also, the generating of the policy model may include calculating a compensation value according to the difference between the target anesthetic state information and the anesthetic state information calculated from the patient's condition changed after the pre-set time interval elapses, after the injection rate of remifentanil and the injection rate of propofol are controlled.

Also, the generating of the policy model may include, based on a plurality of compensation values calculated from changes in different pieces of anesthetic state information, from arbitrary anesthetic state information, calculating an expected value, according to the plurality of compensation values matched to changes in anesthetic state information in a process where a time interval pre-set for a change in the anesthetic state information elapses several times.

Also, the generating of the policy model may include generating the policy model according to a drug injection rate in a change in the anesthetic state information matched to a compensation value selected so that the expected value is calculated as a maximum value.

### Advantageous Effects

According to an aspect of the present disclosure, because a drug injection control device and method using reinforcement learning is provided, a patient's anesthetic state may be stably maintained during surgery by using reinforcement learning.

### DESCRIPTION OF DRAWINGS

FIG. 1 is a schematic view illustrating a drug injection control device, according to an embodiment of the present disclosure.

FIG. 2 is a control block diagram illustrating a drug injection control device, according to an embodiment of the present disclosure.

FIG. 3 is a block diagram illustrating a process by which a policy model training unit of FIG. 2 generates a policy model.

FIG. 4 is a block diagram illustrating a process by which a prediction model training unit of FIG. 2 generates a prediction model.

FIG. 5 is a flowchart illustrating a drug injection control method, according to an embodiment of the present disclosure.

FIG. 6 is a detailed flowchart illustrating an operation of generating a policy model of FIG. 5.

### BEST MODE

In the following detailed description, reference is made to the accompanying drawings that show, by way of illustration, specific embodiments in which the present disclosure may be practiced. These embodiments are described in sufficient detail to enable one of ordinary skill in the art to embody and practice the present disclosure. It is to be understood that the various embodiments of the present disclosure, although different, are not necessarily mutually exclusive. For example, a particular feature, structure, or characteristic described herein in connection with one embodiment may be implemented in other embodiments without departing from the spirit and scope of the present disclosure. In addition, it is to be understood that the location or arrangement of individual elements in each disclosed embodiment may be changed without departing from the spirit and scope of the present disclosure. The following detailed description is, therefore, not to be taken in a limiting sense, and the scope of the present disclosure is defined only by the appended claims, appropriately interpreted, along with the full range of equivalents to which the claims are entitled. In the drawings, like numerals denote the same or similar functions throughout the various aspects.

Hereinafter, preferred embodiments of the present disclosure will be described in more detail with reference to the drawings.

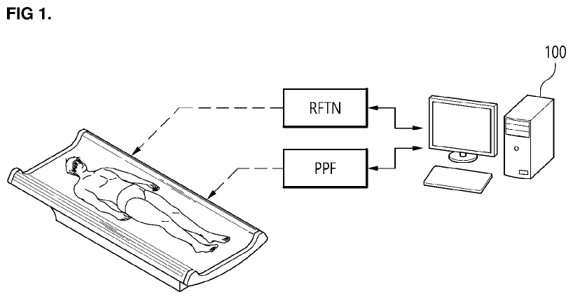

FIG. 1 is a schematic view illustrating a drug injection control device, according to an embodiment of the present disclosure.

A drug injection control device 100 may be provided to control an injection rate of a drug injected into a patient so that the patient's anesthetic state during surgery is maintained constant.

In this case, the patient's anesthetic state may be measured by using bispectral index (BIS), and in general, the BIS of the patient anesthetized during surgery may be recommended to be maintained at 50.

To this end, the drug injection control device 100 is provided to maintain the BIS of the patient constant by adjusting an injection rate of remifentanil and an injection rate of propofol injected into the patient.

The drug injection control device 100 may calculate anesthetic state information of the patient. The anesthetic state information may refer to the BIS calculated or measured from the patient's condition.

In this case, the drug injection control device 100 may measure an anesthetic depth of the patient by using an anesthetic depth monitoring device or an anesthetic depth measuring device provided to measure the patient's anesthetic state, and in this case, the anesthetic state information of the patient may refer to an anesthetic depth of the patient measured by the anesthetic depth monitoring device or the anesthetic depth measuring device.

To this end, the drug injection control device 100 may calculate the patient's fat-free mass according to a height and a weight of the patient, and the drug injection control device 100 may calculate an effect-site concentration and a plasma concentration, based on the patient's fat-free mass and a drug model that is pre-provided for a drug injected into the patient.

Equations 1 to 5 may be equations used to calculate the effect-site concentration and the plasma concentration of the patient.

$${lbm}_{male} = 1.1 \cdot weight - 128 \cdot \frac{weight}{height} \qquad [ Equation 1 ]$$

$${lbm}_{female} = 1.07 \cdot weight - 148 \cdot \frac{weight}{height}$$

Here, lbm_male may denote a male's fat-free mass, and lbm_female may denote a female's fat-free mass. Also, Weight may denote a patient's weight, and Height may denote the patient's height.

$${Vol}_{1}^{P P F} = {h}_{1} , \qquad [ Equation 2 ]$$

$${Vol}_{2}^{P P F} = {h}_{2} - {h}_{3} ( age - {h}_{4} ) ,$$

$${Vol}_{3}^{P P F} = {h}_{5} ,$$

$${C}_{l 1}^{P P F} = {h}_{6} + {h}_{7} ( weight - {h}_{8} ) - {h}_{9} ( lbm - {h}_{10} ) + {h}_{11} ( height - {h}_{12} ) ,$$

$${C}_{l 2}^{P P F} = {h}_{13} - {h}_{14} ( age - {h}_{15} ) ,$$

$${C}_{l 3}^{P P F} = {h}_{16} ,$$

$${k}_{10}^{P P F} = \frac{{C}_{l 1}^{P P F}}{{Vol}_{1}^{P P F}} ,$$

$${k}_{12}^{P P F} = \frac{{C}_{l 2}^{P P F}}{{Vol}_{1}^{P P F}} ,$$

$${k}_{13}^{P P F} = \frac{{C}_{l 3}^{P P F}}{{Vol}_{1}^{P P F}} ,$$

$${k}_{21}^{P P F} = \frac{{C}_{l 2}^{P P F}}{{Vol}_{2}^{P P F}} ,$$

$${k}_{31}^{P P F} = \frac{{C}_{l 3}^{P P F}}{{Vol}_{3}^{P P F}} ,$$

$${k}_{e 0}^{P P F} = {h}_{17} .$$

Here, PPF may denote propofol, h_1 to h_17 may denote variable values by a Schnider model that is a drug model defined for propofol, lbm may denote a patient's fat-free mass, and age may denote the patient's age. Also, Vol_1\hat{ }PPF to Vol_3\hat{ }PPF may denote the amount of drug for each component of propofol, C_I1\hat{ }PPF to C_I3\hat{ }PPF may denote a rate at which the drug in the body is removed for each component of propofol, and k_ij\hat{ }PPF may denote a variable representing a change or modification of the drug in the body for each component of propofol.

TABLE 1

| Drug Parameter | Values |
|---|---|
| Propofol | Schinider |
| h₁ | 4.27 |
| h₂ | 18.9 |
| h₃ | 0.391 |
| h₄ | 53 |
| h₅ | 238 |
| h₆ | 1.89 |
| h₇ | 0.0456 |
| h₈ | 77 |
| h₉ | 0.0681 |
| h₁₀ | 59 |
| h₁₁ | 0.0264 |
| h₁₂ | 177 |
| h₁₃ | 1.29 |
| h₁₄ | 0.024 |
| h₁₅ | 53 |
| h₁₆ | 0.836 |
| h₁₇ | 0.456 |

Table 1 shows a variable for each component of propofol in an arbitrary patient condition according to a Schnider model.

In this case, the Schnider model is known in ‘The influence of method of administration and covariates on the pharmacokinetics of propofol in adult volunteers (T. Schnider, C. Minto, P. Gambus, C. Andresen, D. Goodale, S. Shafer, and E. Youngs, Anesthesiology, vol. 88, no. 5, pp. 1170-1182, 1998.)’, and thus, a detailed description will be omitted.

$${Vol}_{1}^{R F T N} = {f}_{1} - {f}_{2} ( age - {f}_{17} ) + {f}_{3} ( lbm - {f}_{18} ) , \qquad [ Equation 3 ]$$

$${Vol}_{2}^{R F T N} = {f}_{4} - {f}_{5} ( age - {f}_{17} ) + {f}_{6} ( lbm - {f}_{18} ) ,$$

$${Vol}_{3}^{R F T N} = {f}_{7} ,$$

$${C}_{l 2}^{R F T N} = {f}_{11} - {f}_{12} ( age - {f}_{17} ) ,$$

$${C}_{l 3}^{R F T N} = {f}_{13} - {f}_{14} ( age - {f}_{17} ) ,$$

$${k}_{10}^{R F T N} = \frac{{C}_{l 1}^{R F T N}}{{Vol}_{1}^{R F T N}} ,$$

$${k}_{12}^{R F T N} = \frac{{C}_{l 2}^{R F T N}}{{Vol}_{1}^{R F T N}} ,$$

$${k}_{13}^{R F T N} = \frac{{C}_{l 3}^{R F T N}}{{Vol}_{1}^{R F T N}} ,$$

$${k}_{21}^{R F T N} = \frac{{C}_{l 2}^{R F T N}}{{Vol}_{2}^{R F T N}} ,$$

$${k}_{31}^{R F T N} = \frac{{C}_{l 3}^{R F T N}}{{Vol}_{3}^{R F T N}} ,$$

$${k}_{e 0}^{R F T N} = {f}_{15} - {f}_{16} ( age - {f}_{17} )$$

Here, RFTN may denote remifentanil, f_1 to f_17 may denote variable values by a Minto model that is a drug model defined for remifentanil, lbm may denote a patient's fat-free mass, and age may denote the patient's age. Also, Vol_1\hat{ }RFTN to Vol_3\hat{ }RFTN may denote the amount of drug for each component of remifentanil, C_I1\hat{ }RFTN to C_I3\hat{ }RFTN may denote a rate at which the drug in the body is removed for each component of remifentanil, and k_ij\hat{ }RFTN may denote a variable representing a change or modification of the drug in the body for each component of remifentanil.

TABLE 2

| Drug Parameter | Values |
|---|---|
| Remifentanil | Minto |
| f₁ | 5.1 |
| f₂ | 0.0201 |
| f₃ | 0.072 |
| f₄ | 9.82 |
| f₅ | 0.0811 |
| f₆ | 0.108 |
| f₇ | 5.42 |
| f₈ | 2.6 |
| f₉ | 0.0162 |
| f₁₀ | 0.0191 |
| f₁₁ | 2.05 |
| f₁₂ | 0.0301 |
| f₁₃ | 0.076 |
| f₁₄ | 0.00113 |
| f₁₅ | 0.595 |
| f₁₆ | 0.007 |
| f₁₇ | 40 |
| f₁₈ | 55 |

Table 2 shows a variable for each component of remifentanil in an arbitrary patient condition according to a Minto Model.

In this case, the Minto model is known in ‘Influence of Age and Gender on the Pharmacokinetics and Pharmacodynamics of remifentanil. I. Model development (C. Minto, T. Schnider, T. Egan, E. Youngs, H. Lemmens, P. Gambus, V. Billard, J. Hoke, K. Moore, D. Hermann et al., Anesthesiology, vol. 86, no. 1, pp. 10-23, 1997.)’, and thus a detailed description will be omitted.

\hat{x}₁(t)=−[k₁₀+k₁₂+k₁₃]x₁+k₂₁x₂(t)+k₃₁x₃(t)+u(t),

\hat{x}₂(t)=k₁₂x₁(t)−k₂₁x₂(t),

x₃(t)=k₁₃x₁(t)−k₃₁x₃(t). [Equation 4]

Here, xi(t) may denote a variable representing the amount of each component of a drug over time, and k_ij may denote a variable representing a change or modification of the drug in the body for each component of the drug.

\hat{x}ₑ(t)=−kₑ₀xₑ(t)+k₁ₑ\hat{x}₁(t),

Cₚ(t)=x₁(t)/Vol₁

Ĉₑ(t)=kₑ₀(Cₚ(t)−Cₑ(t)). [Equation 5]

Here, x_e(t) may denote a variable representing the amount of a drug at an effect site of the drug over time, C_p(t) may denote a plasma concentration of the drug, and C_e(t) may denote an effect-site concentration indicating a concentration at the effect site of the drug.

Accordingly, the drug injection control device 100 may calculate an effect-site concentration and a plasma concentration, based on the patient's fat-free mass and a drug model that is pre-provided for a drug injected into the patient, according to Equations 1 to 5.

In this case, the drug injection control device 100 may calculate an effect-site concentration and a plasma concentration for propofol or remifentanil, and to this end, the drug injection control device 100 may receive information such as the patient's age, gender, weight, and height.

The drug injection control device 100 may calculate anesthetic state information to indicate the patient' condition according to the calculated effect-site concentration and the calculated plasma concentration.

Equation 6 may be an equation for calculating anesthetic state information from an effect-site concentration and a plasma concentration.

BIS(t)=98.0·(1+Cₑ^{PPF}(t)/4.47+Cₑ^{RFTN}(t)/19.3)^{-1.43}+∈ [Equation 6]

Here, BIS may denote a BIS value of a patient used as anesthetic state information. C_e\hat{ }PPF may denote an effect-site concentration calculated for propofol, C_e\hat{ }RFTN may denote an effect-site concentration calculated for remifentanil, and Epsilon may denote Gaussian noise for the BIS.

In this regard, the drug injection control device 100 may calculate anesthetic state information of the patient according to a pre-set time interval.

The drug injection control device 100 may set target anesthetic state information pre-set for the anesthetic state information of the patient, the drug injection control device 100 may calculate a compensation value according to a change in anesthetic state information due to a drug injection rate set so that the anesthetic state information calculated according to the patient' condition follows the target anesthetic state information, and the drug injection control device 100 may generate a policy model by learning the calculated compensation value.

Target anesthetic state information may refer to a value of anesthetic state information provided to maintain a stable anesthetic state of a patient, and for example, the target anesthetic state information may be set to 50 of BIS that ranges between 0 and 100.

Also, a drug injection rate may include an injection rate of propofol injected into a patient and an injection rate of remifentanil injected into the patient, and in this case, the injection rate of propofol and the injection rate of remifentanil injected into the patient may be set to be different from each other.

Equation 7 may be an equation representing a compensation value calculated according to a change in anesthetic state information.

R(sₜ,aₜ)=1/(|gₜ−BIS(t)|+α)=(|BISₑᵣᵣₒᵣ^{hist}(t)|+α) [Equation 7]

Here, R may denote a compensation value calculated according to a change in anesthetic state information due to an injection rate of a drug and a patient's condition, s_t may denote a state variable provided to indicate anesthetic state information of the patient and the injection rate of the drug, and a_t may denote an operation variable provided to indicate the injection rate of the drug controlled by the drug injection control device 100.

Also, g_t may denote target anesthetic state information provided so that the patient maintains a stable anesthetic state, anesthetic state information may denote anesthetic state information calculated from the patient's condition, and BIS_error\hat{ }hist may denote a change amount of anesthetic state information changed according to a pre-set time interval. Also, Alpha may denote a pre-set maximum compensation value.

Accordingly, the drug injection control device 100 may calculate a compensation value that is a reciprocal of a result value obtained by adding a pre-set maximum compensation value to an absolute value of a difference between the target anesthetic state information and the anesthetic state information calculated from the patient's condition.

In this regard, a state variable may include a difference between the anesthetic state information calculated from the patient's condition and the target anesthetic state information, an injection rate of remifentanil injected into the patient during a pre-set time interval, and an injection rate of propofol injected into the patient during the pre-set time interval.

Also, an operation variable may be provided to indicate an injection rate of a drug injected into the patient during the pre-set time interval.

For example, the drug injection control device 100 may set the pre-set time interval to 10 seconds, the drug injection control device 100 may set a maximum amount of propofol injected into the patient for 10 seconds to 27.8 mg, and the drug injection control device 100 may set a maximum amount of remifentanil injected into the patient for 10 seconds to 27.8 ug. In this case, an operation variable for propofol may be set to an injection rate at which 0 to 27.8 mg of propofol is injected per 10 seconds, and an operation variable for remifentanil may be set to an injection rate at which 0 and 27.8 ug of remifentanil is injected per 10 seconds.

Accordingly, a compensation value, depending on the difference between the anesthetic state information calculated from the patient's condition and the target anesthetic state information, the injection rate of remifentanil injected into the patient during the pre-set time interval, and the injection rate of propofol injected into the patient during the pre-set time interval, may be a value provided to represent compensation for a result of controlling the injection rate of remifentanil injected into the patient and the injection rate of propofol injected into the patient.

In this case, a result of controlling an injection rate of a drug may refer to a state according to a state variable after the pre-set time interval elapses after the injection rate of remifentanil and the injection rate of propofol are controlled.

In other words, the drug injection control device 100 may calculate a compensation value according to the difference between the target anesthetic state information and the anesthetic state information calculated from the patient's condition changed after the pre-set time interval elapses, after the injection rate of remifentanil and the injection rate of propofol are controlled.

Accordingly, the drug injection control device 100 may calculate, based on a plurality of compensation values calculated from changes in different pieces of anesthetic state information, from arbitrary anesthetic state information, an expected value according to a plurality of compensation values matched to changes in anesthetic state information in a process where a time interval pre-set for a change in anesthetic state information elapses several times.

Equation 8 is an equation for calculating an expected value from a compensation value.

[symbol]

(π_{θ})=∫_{τ}P(τ|π_{θ})R(τ)= [symbol] _{τ˜π}_{_{θ}}[R(τ)] [Equation 8]

Here, J(Phi_Theta) may denote an expected value provided to indicate a compensation value calculated when a time interval pre-set for a change in anesthetic state information elapses several times, P(Tau|Phi_Theta) may denote a probability value provided to indicate a probability that an arbitrary operation variable occurs in an arbitrary state variable, and R(Tau) may denote a compensation value.

Accordingly, when a time interval pre-set for a change in anesthetic state information elapses several times, the drug injection control device 100 may calculate an expected value represented by a sum of a product of a compensation value and a probability value at each time interval.

In this regard, Equation 9 is an equation for calculating a probability value.

P(τ|π_{θ}) [symbol] ρ(s₀)Πₜ₌₀^{T-1}P(sₜ₊₁|sₜ,aₜ)π_{θ}(aₜ|sₜ,gₜ) [Equation 9]

Here, P(Tau|Phi_Theta) may denote a probability value provided to indicate a probability that an arbitrary operation variable occurs at an arbitrary state variable, and Rho(S_0) may denote an initial state variable provided to include a difference between anesthetic state information initially measured from a patient and target anesthetic state information, an injection rate of remifentanil initially injected into the patient, and an injection rate of propofol initially injected into the patient. Also, P(S_t+1|s_t, a_t) may denote a probability value in which a state variable changes to the arbitrary state variable after a time interval pre-set by the arbitrary state variable and the arbitrary operation variable elapses, and Phi_Theta(a_t|s_t, g_t) may denote a policy model provided to select a specific operation variable according to the arbitrary state variable and the target anesthetic state information.

Accordingly, the drug injection control device 100 may calculate a probability value obtained by multiplying an initial state variable by a value obtained by multiplying a product of a policy model and a probability value in which a state variable changes at different time intervals.

In this case, the drug injection control device 100 may generate a policy model according to a drug injection rate in a change in anesthetic state information matched to a compensation value selected so that an expected value is calculated as a maximum value.

In other words, the drug injection control device 100 may select a compensation value so that an expected value is calculated as a maximum value by learning a compensation value according to a change in anesthetic state information due to an arbitrary drug injection rate, and the drug injection control device 100 may generate a policy model so that a drug injection rate matched to the selected compensation value is represented as an operation variable.

The drug injection control device 100 may generate a policy model by learning a compensation value from a plurality of data sets pre-provided to include a change in anesthetic state information according to a time interval pre-set for an arbitrarily set drug injection rate.

In this regard, the drug injection control device 100 may generate a policy by learning compensation according to a changing state by performing an arbitrary operation in an arbitrary state, and may generate a policy model by using reinforcement learning that selects an operation performed in an arbitrary state according to the generated policy.

The drug injection control device 100 may generate a prediction model, by learning a change in anesthetic state information according to a drug injection rate.

To this end, for the information about the patient's age, gender, weight, and height, the drug injection control device 100 may learn a change in the difference between the target anesthetic state information and the anesthetic state information calculated from the patient's condition according to the pre-set time interval, the injection rate of remifentanil injected into the patient during the pre-set time interval, and the injection rate of propofol injected into the patient during the pre-set time interval.

In this case, the drug injection control device 100 may be provided to input the information about the patient's age, gender, weight, and height and the change in the state variable according to the pre-set time interval into a deep neural network (DNN) or a long short-term memory model (LSTM).

The DNN is a network in which a plurality of hidden layers are provided between an input layer and an output layer and feature information is output from information input to the input layer, and the LSTM is a network in which information is received in a temporal sequence and the amount of output information is adjusted based on previously input information and currently input information.

Accordingly, the drug injection control device 100 may input information output from the DNN or the LSTM into one DNN and may fuse the information, and in this case, the drug injection control device 100 may be provided to input information output from the DNN used for information fusion into another DNN provided to output expected anesthetic state information.

Accordingly, the drug injection control device 100 may generate expected anesthetic state information by using the information such as the patient's age, gender, weight, and height, and the change in the difference between the target anesthetic state information and the anesthetic state information calculated from the patient's condition according to the pre-set time interval, the injection rate of remifentanil injected into the patient during the pre-set time interval, and the injection rate of propofol injected into the patient during the pre-set time interval.

The drug injection control device 100 may set a drug injection rate from anesthetic state information, based on the policy model.

In this case the drug injection control device 100 may control the injection rate of remifentanil and the injection rate of propofol according to the difference between the target anesthetic state information and the anesthetic state information calculated from the patient's condition, the injection rate of remifentanil injected into the patient during the pre-set time interval, and the injection rate of propofol injected into the patient during the pre-set time interval.

In other words, the drug injection control device 100 may select an operation variable set by the policy model according to a current state variable, and may control an injection rate of a drug injected into the patient according to the operation variable.

The drug injection control device 100 may predict expected anesthetic state information from the set drug injection rate and a previously set drug injection rate, based on the prediction model.

In this case, the drug injection control device 100 may output the predicted expected anesthetic state information as a graph or a numerical value so that a user identifies the predicted expected anesthetic state information, and to this end, the drug injection control device 100 may include a separate display device.

The drug injection control device 100 may calculate a compensation value according to the patient's condition according to expected anesthetic state information from the patient's condition according to the anesthetic state information calculated from the patient's condition by using the predicted expected anesthetic state information, and in this case, the drug injection control device 100 may generate a policy model by learning the compensation value based on the expected anesthetic state information.

Also, the drug injection control device 100 may measure an anesthetic depth of the patient by using an anesthetic depth monitoring device or an anesthetic depth measuring device provided to measure the patient's anesthetic state, and in this case, the drug injection control device 100 may generate a policy model according to the anesthetic depth measured from the patient and a target anesthetic depth set so that the patient's anesthetic state is stably maintained.

A device for measuring the patient's anesthetic state such as the anesthetic depth monitoring device or the anesthetic depth measuring device may be a device according to any instrument, device, and method using well-known technology.

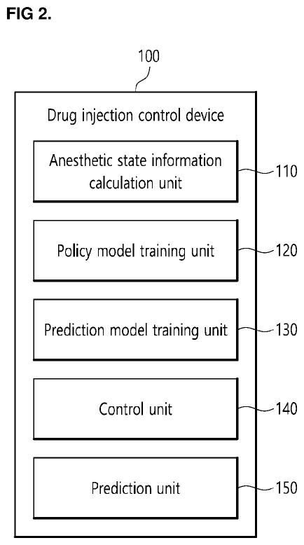

FIG. 2 is a control block diagram illustrating a drug injection control device, according to an embodiment of the present disclosure.

The drug injection control device 100 may include an anesthetic state information calculation unit 110, a policy model training unit 120, a prediction model training unit 130, a control unit 140, and a prediction unit 150.

The anesthetic state information calculation unit 110 may calculate anesthetic state information of a patient, and in this regard, the anesthetic state information calculation unit 110 may receive information such as the patient's age, gender, weight, and height.

To this end, the anesthetic state information calculation unit 110 may calculate the patient's fat-free mass according to the patient's height and weight, and the anesthetic state information calculation unit 110 may calculate an effect-site concentration and a plasma concentration, based on the patient's fat-free mass and a drug model pre-provided for a drug injected into the patient. In this case, the anesthetic state information calculation unit 110 may calculate an effect-site concentration and a plasma concentration for propofol or remifentanil.

Accordingly, the anesthetic state information calculation unit 110 may calculate anesthesia state information to indicate the patient's condition according to the calculated effect-site concentration and the calculated plasma concentration, and in this case, the anesthetic state information calculation unit 110 may calculate the anesthetic state information of the patient according to a pre-set time interval.

The policy model training unit 120 may set target anesthetic state information pre-set for the anesthetic state information of the patient, the policy model training unit 120 may calculate a compensation value according to a change in anesthetic state information due to a drug injection rate set so that the anesthetic state information calculated according to the patient's condition follows the target anesthetic state information, and the policy model training unit 120 may generate a policy model by learning the calculated compensation value.

In this case, the policy model training unit 120 may calculate a compensation value that is a reciprocal of a result value obtained by adding a pre-set maximum compensation value to an absolute value of a difference between the target anesthetic state information and the anesthetic state information calculated from the patient's condition.

In other words, the policy model training unit 120 may calculate a compensation value according to the difference between the target anesthetic state information and anesthetic state information calculated from the patient's condition changed after the pre-set time interval elapses, after an injection rate of remifentanil and an injection rate of propofol are controlled.

Also, the policy model training unit 120 may calculate, based on a plurality of compensation values calculated from changes in different pieces of anesthetic state information, from arbitrary anesthetic state information, an expected value according to a plurality of compensation values matched to changes in anesthetic state information in a process where a time interval pre-set for a change in anesthetic state information elapses several times.

When a time interval pre-set for a change in anesthetic state information elapses several times, the policy model training unit 120 may calculate an expected value represented by a sum of a product of a compensation value and a probability value at each time interval.

In this regard, the policy model training unit 120 may calculate a probability value obtained by multiplying an initial state variable by a value obtained by multiplying a product of a policy model and a probability value in which a state variable changes at different time intervals.

In this case, the policy model training unit 120 may generate a policy model according to a drug injection rate in a change in anesthetic state information matched to a compensation value selected so that an expected value is calculated as a maximum value.

In other words, the policy model training unit 120 may select a compensation value so that an expected value is calculated as a maximum value by learning a compensation value according to a change in anesthetic state information due to an arbitrary drug injection rate, and the policy model training unit 120 may generate a policy model so that a drug injection rate for a change in anesthetic state information matched to the selected compensation value is represented as an operation variable.

The policy model training unit 120 may generate a policy model by learning a compensation value from a plurality of data sets pre-provided to include a change in anesthetic state information according to a time interval pre-set for an arbitrarily set drug injection rate.

In this regard, the policy model training unit 120 may generate a policy by learning compensation according to a changing state by performing an arbitrary operation in an arbitrary state, and may generate a policy model by using reinforcement learning that selects an operation performed in an arbitrary state according to the generated policy.

The policy model training unit 120 may calculate a compensation value according to the patient's condition according to expected anesthetic state information from the patient's condition according to anesthetic state information calculated from the patient's condition, by using predicted expected anesthetic state information, and in this case, the policy model training unit 120 may generate a policy model by learning the compensation value based on the expected anesthetic state information.

The prediction model training unit 130 may generate a prediction model, by learning a change in anesthetic state information according to a drug injection rate.

To this end, for information such as the patient's age, gender, weight, and height, the prediction model training unit 130 may learn a change in the difference between the target anesthetic state information and the anesthetic state information calculated from the patient's condition according to the pre-set time interval, the injection rate of remifentanil injected into the patient during the pre-set time interval, and the injection rate of propofol injected into the patient during the pre-set time interval.

The control unit 140 may set a drug injection rate from anesthetic state information, based on the policy model.

In this case, the control unit 140 may control the injection rate of remifentanil and the injection rate of propofol according to the difference between the target anesthetic state information and the anesthetic state information calculated from the patient's condition, the injection rate of remifentanil injected into the patient during the pre-set time interval, and the injection rate of propofol injected into the patient during the pre-set time interval.

In other words, the control unit 140 may select an operation variable set by the policy model according to a current state variable, and may control an injection rate of a drug injected into the patient according to the operation variable.

The prediction unit 150 may predict expected anesthetic state information from the set drug injection rate and a previously set drug injection rate, based on the prediction model.

In this case, the prediction unit 150 may output the predicted expected anesthetic state information as a graph or a numeral value so that a user identifies the predicted expected anesthetic state information, and to this end, the drug injection control device 100 may include a separate display device.

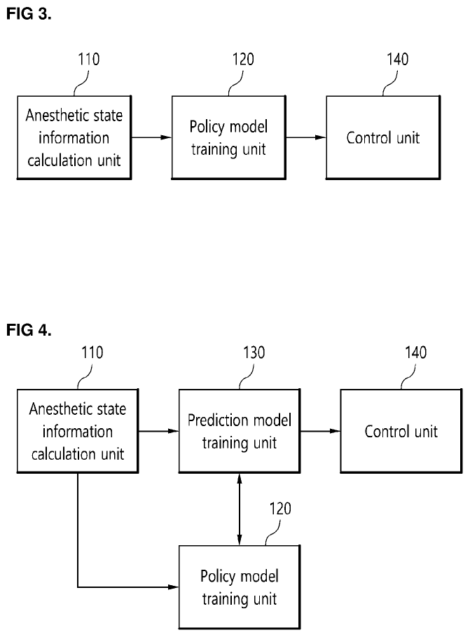

FIG. 3 is a block diagram illustrating a process by which a policy model training unit of FIG. 2 generates a policy model.

Referring to FIG. 3, the anesthetic state information calculation unit 110 may calculate anesthetic state information of a patient, and in this regard, the anesthetic state information calculation unit 110 may receive information such as the patient's age, gender, weight, and height.

To this end, the anesthetic state information calculation unit 110 may calculate the patient's fat-free mass according to the patient's height and weight, and the anesthetic state information 110 may calculate an effect-site concentration and a plasma concentration, based on the patient's fat-free mass and a drug model pre-provided for a drug injected into the patient. In this case, the anesthetic state information calculation unit 110 may calculate an effect-site concentration and a plasma concentration for propofol or remifentanil.

Accordingly, the anesthetic state information calculation unit 110 may calculate anesthetic state information to indicate the patient's condition according to the calculated effect-site concentration and the calculated plasma concentration, and in this case, the anesthetic state information calculation unit 110 may calculate the anesthetic state information of the patient according to a pre-set time interval.

In this case, the policy model training unit 120 may set target anesthetic state information pre-set for the anesthetic state information of the patient, and the policy model training unit 120 may generate a policy model by learning a compensation value according to a change in anesthetic state information due to a drug injection rate set so that the anesthetic state information of the patient follows the target anesthetic state information.

In this case, the policy model training unit 120 may calculate a compensation value that is a reciprocal of a result value obtained by adding a pre-set maximum compensation value to an absolute value of a difference between the target anesthetic state information and the anesthetic state information calculated form the patient's condition.

In other words, the policy model training unit 120 may calculate a compensation value according to the difference between the target anesthetic state information and the anesthetic state information calculated from the patient's condition changed after the pre-set time interval elapses, after an injection rate of remifentanil and an injection rate of propofol are controlled.

Also, the policy model training unit 120 may calculate, based on a plurality of compensation values calculated from changes in different pieces of anesthetic state information, from arbitrary anesthetic state information, an expected value according to a plurality of compensation values matched to changes in anesthetic state information in a process in which a time interval pre-set for a change in anesthetic state information elapses several times.

When a time interval pre-set for a change in anesthetic state information elapses several times, the policy model training unit 120 may calculate an expected value represented by a sum of a product of a compensation value and a probability value at each time interval.

In this regard, the policy model training unit 120 may calculate a probability value obtained by multiplying an initial state variable by a value obtained by multiplying a product of a policy model and a probability value in which a state variable changes at different time intervals.

In this case, the policy model training unit 120 may generate a policy model according to a drug injection rate in a change in anesthetic state information matched to a compensation value selected so that an expected value is calculated as a maximum value.

In other words, the policy model training unit 120 may select a compensation value so that an expected value is calculated as a maximum value by learning a compensation value according to a change in anesthetic state information due to an arbitrary drug injection rate, and the policy model training unit 120 may generate a policy model so that a drug injection rate for a change in anesthetic state information matched to the selected compensation value is represented as an operation variable.

The policy model training unit 120 may generate a policy model by learning a compensation value from a plurality of data sets pre-provided to include a change in anesthetic state information according to a time interval pre-set for an arbitrarily set drug injection rate.

Accordingly, the control unit 140 may set a drug injection rate from anesthetic state information, based on the policy model.

In this case, the control unit 140 may control the injection rate of remifentanil and the injection rate of propofol according to the difference between the target anesthetic state information and the anesthetic state information calculated from the patient's condition, the injection rate of remifentanil injected into the patient during the pre-set time interval, and the injection rate of propofol injected into the patient during the pre-set time interval.

In other words, the control unit 140 may select an operation variable set by the policy model according to a current state variable, and may control an injection rate of a drug injected into the patient according to the operation variable.

FIG. 4 is a block diagram illustrating a process by which a prediction model training unit of FIG. 2 generates a prediction model.

Referring to FIG. 4, the anesthetic state information calculation unit 110 may calculate anesthetic state information of a patient, and in this regard, the anesthetic state information calculation unit 110 may receive information such as the patient's age, gender, weight, and height.

To this end, the anesthetic state information calculation unit 110 may calculate the patient's fat-free mass according to the patient's height and weight, and the anesthetic state information calculation unit 110 may calculate an effect-site concentration and a plasma concentration, based on the patient's fat-free mass and a drug model pre-provided for a drug injected into the patient. In this case, the anesthetic state information calculation unit 110 may calculate an effect-site concentration and a plasma concentration for propofol or remifentanil.

Accordingly, the anesthetic state information calculation unit 110 may calculate anesthesia state information to indicate the patient's condition according to the calculated effect-site concentration and the calculated plasma concentration, and in this case, the anesthetic state information calculation unit 110 may calculate the anesthetic state information of the patient according to a pre-set time interval.

In this case, the prediction model training unit 130 may generate a prediction model, by learning anesthetic state information that changes according to a change in a drug injection rate.

To this end, for the information such as the patient's age, gender, weight, and height, the prediction model training unit 130 may learn a change in a difference between target anesthetic state information and anesthetic state information calculated from the patient's condition according to a pre-set time interval, an injection rate of remifentanil injected into the patient during the pre-set time interval, and an injection rate of propofol injected into the patient during the pre-set time interval.

Accordingly, the prediction unit 150 may predict expected anesthetic state information from a set drug injection rate and a previously set drug injection rate, based on the prediction model.

In this case, the prediction unit 150 may output the predicted expected anesthetic state information as a graph or a numerical value so that a user identifies the predicted expected anesthetic state information, and to this end, the drug injection control device 100 may include a separate display device.

The policy model training unit 120 may calculate a compensation value according to the patient's condition according to expected anesthetic state information from the patient's condition according to the anesthetic state information calculated from the patient's condition, by using the predicted expected anesthetic state information, and in this case, the policy model training unit 120 may generate a policy model by learning the compensation value based on the expected anesthetic state information.

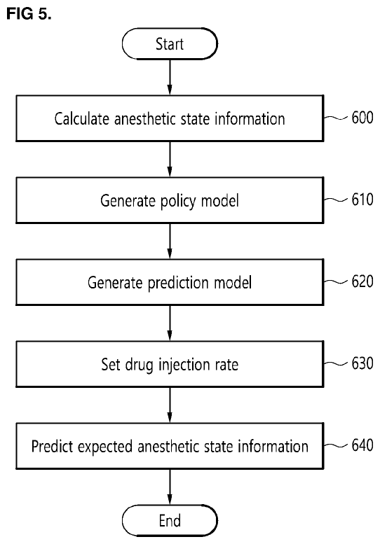

FIG. 5 is a flowchart illustrating a drug injection control method, according to an embodiment of the present disclosure.

A drug injection control method according to an embodiment of the present disclosure is performed by substantially the same configuration as the drug injection control device 100 of FIG. 1, and thus, the same elements as those of the drug injection control device 100 of FIG. 1 are denoted by the same reference numerals and a repeated description thereof will be omitted.

The drug injection control method may include an operation 600 of calculating anesthetic state information, an operation 610 of generating a policy model, an operation 620 of generating a prediction model, an operation 630 of setting a drug injection rate, and an operation 640 of predicting expected anesthetic state information.

The operation 600 of calculating anesthetic state information may be an operation in which the anesthetic state information calculation unit 110 calculates anesthetic state information of a patient.

The operation 610 of generating a policy model may be an operation in which the policy model training unit 20 sets target anesthetic state information pre-set for anesthetic state information, calculates a compensation value according to a change in anesthetic state information due to a drug injection rate set so that the calculated anesthetic state information follows the target anesthetic state information, and generates a policy model by learning the compensation value.

The operation 620 of generating a prediction model may be an operation in which the prediction model training unit 130 generates a prediction model by learning a change in anesthetic state information according to a drug injection rate.

The operation 630 of setting a drug injection rate may be an operation in which the control unit 140 sets a drug injection rate from the anesthetic state information, based on the policy model.

The operation 640 of predicting expected anesthetic state information may be an operation in which the prediction unit 150 predicts expected anesthetic state information from the set drug injection rate and a previously set drug injection rate based on the prediction model.

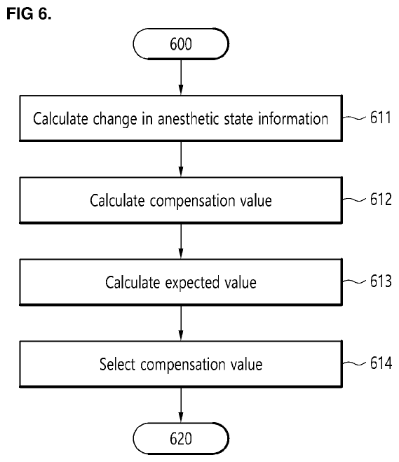

FIG. 6 is a detailed flowchart illustrating an operation of generating a policy model of FIG. 5.

The operation 610 of generating a policy model may include an operation 611 of calculating a change in anesthetic state information, an operation 612 of calculating a compensation value, an operation 613 of calculating an expected value, and an operation 614 of selecting a compensation value.

The operation 611 of calculating a change in anesthetic state information may be an operation in which, regarding a difference between target anesthetic state information and anesthetic state information calculated from a patient's condition, the policy model training unit 120 calculates a change in anesthetic state information according to the difference between the target anesthetic state information and the anesthetic state information calculated from the patient's condition changed after a pre-set time interval elapses, after an injection rate of remifentanil and an injection rate of propofol are controlled.

The operation 612 of calculating a compensation value may be an operation in which the policy model training unit 120 calculates a compensation value that is a reciprocal of a result value obtained by adding a pre-set maximum compensation value to an absolute value of the difference between the target anesthetic state information and the anesthetic state information calculated from the patient's condition.

In other words, the operation 612 of calculating a compensation value may be an operation in which the policy model training unit 120 calculates a compensation value according to the difference between the target anesthetic state information and anesthetic state information calculated from the patient's condition changed after the pre-set time interval elapses, after the injection rate of remifentanil and the injection rate of propofol are controlled.

The operation 613 of calculating an expected value may be an operation in which the policy model training unit 120 calculates, based on a plurality of compensation values calculated from changes in different pieces of anesthetic state information, from arbitrary anesthetic state information, an expected value according to a plurality of compensation values matched to changes in anesthetic state information in a process where a time interval pre-set for a change in anesthetic state information elapses several times.

The operation 614 of selecting a compensation value may be an operation in which the policy model training unit 120 selects a compensation value so that an expected value is calculated as a maximum value, by learning a compensation value according to a change in anesthetic state information due to an arbitrary drug injection rate, and thus, the policy model training unit 120 may generate a policy model so that a drug injection rate for a change in anesthetic state information matched to the selected compensation value is represented as an operation variable.

Although the present disclosure has been described with reference to embodiments, it will be understood by one of ordinary skill in the art that various modifications and changes may be made without departing from the spirit and cope of the present disclosure defined by the following claims.

### DESCRIPTION OF MAIN ELEMENTS

100: drug injection control device
110: anesthetic state information calculation unit
120: policy model training unit
130: prediction model training unit
140: control unit
150: prediction unit

1. A drug injection control device comprising:
an anesthetic state information calculation unit configured to calculate anesthetic state information of a patient;
a policy model training unit configured to set target anesthetic state information pre-set for the anesthetic state information, calculate a compensation value according to a change in the anesthetic state information due to a drug injection rate set so that the anesthetic state information follows the target anesthetic state information, and generate a policy model by learning the compensation value;
a prediction model training unit configured to generate a prediction model, by learning the change in the anesthetic state information according to the drug injection rate;
a control unit configured to set the drug injection rate from the anesthetic state information, based on the policy model; and
a prediction unit configured to predict expected anesthetic state information from the set drug injection rate and a previously set drug injection rate, based on the prediction model.

2. The drug injection control device according to claim 1, wherein the anesthetic state information calculation unit is configured to calculate an effect-site concentration and a plasma concentration based on the patient's fat-free mass and a drug model pre-provided for a drug injected into the patient, and calculate the anesthetic state information to indicate the patient's condition according to the effect-site concentration and the plasma concentration.

3. The drug injection control device according to claim 1, wherein the control unit is configured to, according to a difference between the anesthetic state information calculated from the patient's condition and the target anesthetic state information, an injection rate of remifentanil injected into the patient during a pre-set time interval, and an injection rate of propofol injected into the patient during the pre-set time interval, control the injection rate of remifentanil and the injection rate of propofol.

4. The drug injection control device according to claim 3, wherein the policy model training unit is configured to calculate a compensation value according to the difference between the target anesthetic state information and the anesthetic state information calculated from the patient's condition changed after the pre-set time interval elapses, after the injection rate of remifentanil and the injection rate of propofol are controlled.

5. The drug injection control device according to claim 1, wherein the policy model training unit is configured to, based on a plurality of compensation values calculated from changes in different pieces of anesthetic state information, from arbitrary anesthetic state information, calculate an expected value according to a plurality of compensation values matched to changes in anesthetic state information in a process where a time interval pre-set for a change in the anesthetic state information elapses several times.

6. The drug injection control device according to claim 5, wherein the policy model training unit is configured to generate the policy model according to a drug injection rate in a change in the anesthetic state information matched to a compensation value selected so that the expected value is calculated as a maximum value.

7. A drug injection control method using a drug injection control device using reinforcement learning, the drug injection control method comprising:
calculating anesthetic state information of a patient;
setting target anesthetic state information pre-set for the anesthetic state information, calculating a compensation value according to a change in the anesthetic state information due to a drug injection rate set so that the anesthetic state information follows the target anesthetic state information, and generating a policy model by learning the compensation value;
generating a prediction model, by learning the change in the anesthetic state information according to the drug injection rate;
setting the drug injection rate from the anesthetic state information, based on the policy model; and
predicting expected anesthetic state information from the set drug injection rate and a previously set drug injection rate, based on the prediction model.

8. The drug injection control method according to claim 7, wherein the calculating of the anesthetic state information comprises calculating an effect-site concentration and a plasma concentration based on the patient's fat-free mass and a drug model pre-provided for a drug injected into the patient, and calculating the anesthetic state information to indicate the patient's condition according to the effect-site concentration and the plasma concentration.

9. The drug injection control method according to claim 7, wherein the setting of the drug injection rate comprises, according to a difference between the anesthetic state information calculated from the patient's condition and the target anesthetic state information, an injection rate of remifentanil injected into the patient during a pre-set time interval, and an injection rate of propofol injected into the patient during the pre-set time interval, controlling the injection rate of remifentanil and the injection rate of propofol.

10. The drug injection control method according to claim 9, wherein the generating of the policy model comprises calculating a compensation value according to the difference between the target anesthetic state information and the anesthetic state information calculated from the patient's condition changed after the pre-set time interval elapses, after the injection rate of remifentanil and the injection rate of propofol are controlled.

11. The drug injection control method according to claim 7, wherein the generating of the policy model comprises, based on a plurality of compensation values calculated from changes in different pieces of anesthetic state information, from arbitrary anesthetic state information, calculating an expected value, according to the plurality of compensation values matched to changes in anesthetic state information in a process where a time interval pre-set for a change in the anesthetic state information elapses several times.

12. The drug injection control method according to claim 11, wherein the generating of the policy model comprises generating the policy model according to a drug injection rate in a change in the anesthetic state information matched to a compensation value selected so that the expected value is calculated as a maximum value.
